(19) United States
(12) Patent Application Publication
(10) Pub. No.: US 2016/0000938 A1
(43) Pub. Date: Jan. 7, 2016

(54) REAGENT FOR DIAGNOSIS OF OSTEOARTHRITIS COMPRISING PEPTIDE PROBE OF APOPEP-1

(71) Applicants: Je Yong CHOI, Daegu (KR); Byung Hun LEE, Daegu (KR); Xiangguo CHE, Daegu (KR); Lianhua CHI, Daegu (KR)

(72) Inventors: Je Yong CHOI, Daegu (KR); Byung Hun LEE, Daegu (KR); Xiangguo CHE, Daegu (KR); Lianhua CHI, Daegu (KR)

(21) Appl. No.: 14/515,840

(22) Filed: Oct. 16, 2014

(30) Foreign Application Priority Data
Oct. 17, 2013 (KR) 10-2013-0124111

(51) Int. Cl. A61K 49/00; G01N 33/558

(52) U.S. Cl. CPC A61K 49/0056; G01N 33/558; G01N 2800/105

(57) ABSTRACT

Disclosed is a reagent for diagnosing osteoarthritis or predicting the prognosis of osteoarthritis, the reagent containing a peptide having the amino acid sequence (CQRPPR) of SEQ ID NO: 1 as an active ingredient. The present peptide can be used to accurately diagnose osteoarthritis in its early stage based on the molecular imaging technique. The present peptide has a small molecular weight, and thus has advantages of fast clearance from the blood, effective permeation into the tissue, low immunogenicity, and low-production cost. Further, the present reagent can diagnose osteoarthritis in its early stage in which the destruction of cartilage is in a reversible phase and thus can be recovered to a normal state, thereby significantly contributing to effective treatment of osteoarthritis.

### TECHNICAL FIELD

The present invention relates to reagent for diagnosis of osteoarthritis comprising peptide probe of ApoPep-1.

### BACKGROUND ART

The current dilemma of osteoarthritis (OA), which affects 630 million people worldwide, is that the existing cure for the disease is only effective during its early development, but early detection of the disease is difficult due to the lack of symptoms, sensitive methods and high cost. During the early stages of OA, structure-modifying OA drugs (SMOADs), such as chondroitin sulfate, enhance tissue turnover and restore the tissue. Disease-modifying OA drugs (DMOADs) are defined to block structural disease progression, possibly by suppressing dominant catabolic enzymes, and ideally improve symptoms and/or function. Despite the research towards prospective DMOADs, there are currently no licensed DMOADs. During the late stages of OA, almost all cartilage is lost and thus, at present, joint replacement is the single regimen (1-3).

Apoptotic cell death at the articulate cartilage is suggested to be an important event for the diagnosis and treatment of OA. Cartilage is made up of articular (superficial), middle (transitional), deep, and calcified zones, which differ in their matrix composition and architecture. Articular chondrocytes are important in maintaining the dynamic equilibrium between synthesis and degradation of the extracellular matrix (4, 5). The articular cartilage is the first to be affected by OA and its destruction can be a warning sign of OA progression (6-8). During the early stages of OA, chondrocyte apoptosis increases in the articular surface and middle zones of the cartilage, probably as a consequence of constant mechanical damage to the joint (9, 10).

Recent studies of cartilage from equine joints have shown that chondrocyte apoptosis is positively correlated with early stages of OA and severity of cartilage damage, suggesting that this process is intrinsically linked to cartilage damage and may be associated with the initiation of cartilage degradation in OA (11). At early stages of OA, the death of chondrocytes starts with apoptosis in the superficial and part of the middle zones of the cartilage, probably as a consequence of a constant mechanical damage in the joint (12-14). More recently, apoptotic cell death has become a focus of interest and was suggested to be an important event in osteoarthritic cartilage degeneration (15). In particular, detection of the early stages of disease is important, as we expect interventions to be more effective earlier rather than later. Early diagnosis of OA is essential for early treatment of OA, which could halt the progression of the disease and prevent irreversible disability (16). However, the early diagnosis of OA is difficult because clinical manifestation signs or typical radiographic changes cannot be observed during the initial stages of OA. Therefore, accurate technology for the early diagnosis of OA is necessary.

However, current OA diagnosis methods have limitations to detect early stage OA or are costly for the general public. The sensitivity of radiography and computed tomography (CT) is insufficient to detect the early onset of OA. Conventional radiography can only examine joint space narrowing and osteophytes, which are characteristics of advanced OA. Magnetic resonance imaging (MRI) provides high-resolution computerized images of internal body tissue, but is limited in its ability to detect articular chondrocyte apoptosis and collagen fiber loss, which are characteristics of the initiation of OA. In addition, the cost for an MRI scan is expensive for the general public to use as a preventative measure. Techniques to diagnose the initiation of OA is necessary to prevent chronic pain, prolonged difficulty in mobility, possible complications due to joint replacement surgery, and increased healthcare costs for the patient and society (17). Apoptotic chondrocyte death has been reported to occur more frequently in OA cartilage than healthy cartilage in humans and has been positively correlated with the severity of cartilage damage in the joint. Apoptosis indicators include exposed cellular phosphatidylserine, dysfunctional mitochondria, activated caspases, fragmented DNA, and disrupted membrane integrity. Annexin V, a 36 kDa protein that binds to phosphatidylserine, is most commonly used and generally considered an early marker of apoptotic cell death. However, annexin V binds to both type II and type X collagen and facilitates the binding of chondrocytes to collagen. Type II collagen is highly expressed in healthy articular cartilage whereas type X collagen is highly expressed in OA cartilage. Therefore, annexin V would not be suitable to distinguish OA cartilage from healthy cartilage. On the other hand, caspase antibodies can also bind to caspase enzymes to detect programmed cell death, but these antibodies have slow binding kinetics, delayed clearance, and the possibility of immunogenicity. These drawbacks require the development of a new method to detect the initiation of OA (18, 19).

Peptides have attractive potential to be used in diagnostic tools because of their many advantages, including rapid binding kinetics and degradation, minimal concern for immunogenicity, high clonal diversity, and attach ability to diverse probes. Therefore, when used for the diagnosis of OA, peptides may safely and accurately detect osteoarthritic cartilage within a shorter time span and be rapidly excreted from or degraded in the body. For instance, ApoPep-1, a six-amino-add CQRPPR peptide, has been reported to bind to histone H1.2 when exposed to the surface of apoptotic cells (20). ApoPep-1 conjugated with fluorescent dye or radioisotopes has been successfully used in in vivo imaging of apoptosis in tumor cells and neurons (20-22).

In these regards, the present inventors hypothesized that ApoPep-1 could be a useful tool for the diagnosis of early stage of OA and assessment of OA progressions and provide clinicians to diagnosis early stage of OA accurately and conveniently. Using destabilization of the medial meniscus (DMM) mouse model of OA (8, 23), the present inventors want to determine whether ApopPep-1 is an available early diagnostic indicator for OA disease. We found that ApoPep-1 binds specifically to articular chondrocyte undergoing apoptosis, suggesting ApoPep-1 could be a useful imaging probe for detecting early stage of OA for clinical diagnosis.

Throughout the entire specification, many papers and patent documents are referenced and their citations are represented. The disclosures of cited papers and patent documents are entirely incorporated by reference into the present specification, and the level of the technical field within which the present invention falls and details of the present invention are explained more clearly.

### DETAILED DESCRIPTION OF THE INVENTION

### Technical Problem

The present inventors have endeavored to develop a peptide probe for diagnosis, capable of accurately diagnosing osteoarthritis in its early stage in which the destruction of cartilage is in a reversible phase, thereby contributing to an effective treatment of this disease. As a result, the present inventors have experimentally verified that the use of a peptide probe having the amino acid sequence of CQRPPR (SEQ ID NO: 1) can lead to an accurate diagnosis of osteoarthritis in its early stage through ex vivo and in vivo imaging, and then completed the present invention.

Accordingly, it is an object of this invention to provide a method for detecting the osteoarthritis status of a subject using the present peptide probe.

Other objects and advantages of the present invention will become apparent from the following detailed description together with the appended claims and drawings.

### Technical Solution

The present inventors have endeavored to develop a peptide probe for diagnosis, capable of accurately diagnosing osteoarthritis in its early stage in which the destruction of cartilage is in a reversible phase, thereby contributing to an effective treatment of this disease. As a result, the present inventors experimentally verified that the use of a peptide probe having the amino acid sequence of CQRPPR (SEQ ID NO: 1) (hereinafter, also referred to as “ApoPep-1” peptide) can lead to an accurate diagnosis of osteoarthritis in its early stage through ex vivo and in vivo imaging, and then completed the present invention. More specifically, the present inventors used a destabilization of the medial meniscus (DMM) mouse, and verified that the ApoPep-1 peptide fluorescence-labeled after DMM surgery can lead to an accurate diagnosis of osteoarthritis in its early stage in the mouse.

Therefore, the ApoPep-1 peptide of the present invention can be useful as a reagent for diagnosing osteoarthritis or determining the prognosis of osteoarthritis. The ApoPep-1 peptide of the present invention was developed as a peptide for targeting apoptotic cells in the prior art, and disclosed to be able to be used as a molecular imaging probe of diseases, such as, tumor, stroke, myocardial infarction, and atherosclerosis (Korean Registration No. 10-0952841).

As used herein, the term “diagnosis” includes determining the susceptibility of a subject to osteoarthritis, determining whether a subject has osteoarthritis at present, determining the prognosis of a subject with osteoarthritis (e.g., identification of the condition of osteoarthritis, determination of the stage of osteoarthritis, or determination of the reactivity of arthritis to treatment), or monitoring the condition of a subject in order to provide information about the efficacy of osteoarthritis treatment. As used herein, the term “prognosis” includes the possibility of osteoarthritis progress, particularly, the prediction in view of the improvement of disease, the regeneration of disease, or the reoccurrence of osteoarthritis. Preferably, the prediction herein refers to the possibility of complete recovery of a patient with osteoarthritis.

As used herein, the term “osteoarthritis (OA)” is one of the oldest and most common diseases among arthritis disease in which inflammation occurs in the joint, and refers to a chronic condition characterized by destruction of joint's cartilage. The cartilage is a site of the joint, which performs a cushion function at the end portion of the bone to facilitate the movement of joint. The destruction of cartilage causes abrasion between adjacent bones, and causes stiffness and pain due to the difficulty in joint movement.

Osteoarthritis may be divided into the following various stages depending on the progress of disease: (i) a stage in which the cartilage loses its elasticity and thus is more easily damaged by an injury or a use thereof; and (ii) a stage in which of the abrasion of the cartilage causes a change in the underlying bone, resulting in the thickening of bones and the generation of cysts, wherein the growth of bone called spurs or osteophytes occur in the end of the bone of the affected joint, causing itching or pain; (iii) a stage in which pieces of the bone or cartilage loosely float in a joint space; and (iv) a stage in which the breakdown of cartilage causes inflammation in a joint membrane or a synovial membrane, and further brings about cytokines and enzymes that damage the cartilage.

As used herein, the term “subject” refers an object of which the condition of osteoarthritis can be detected by using the peptide probe of the present invention, specifically a mammal, more specifically, a human or an animal other than a human, and still more specifically, a human, a mouse, a rat, a hamster, a rabbit, a guinea pig, a dog, a pig, a cow, or a primate, but is not limited thereto.

As used herein, the term “normal” refers to a generally healthy state without particular disease, specifically, a state without osteoarthritis, and more specifically, a state in which the subject or chondrocytes do not have osteoarthritis.

The term refers an object of which the condition of osteoarthritis can be detected by using the peptide probe of the present invention, specifically a mammal, more specifically, a human or an animal other than a human, and still more specifically, a human, a mouse, a rat, a hamster, a rabbit, a guinea pig, a dog, a pig, a cow, or a primate, but is not limited thereto.

According to a specific embodiment of the present invention, the diagnosis of the osteoarthritis is the diagnosis of osteoarthritis in its early stage. The ApoPep-1 peptide of the present invention can accurately diagnose osteoarthritis in its early stage.

As used herein, the term “osteoarthritis in its early stage” refers to osteoarthritis in a reversible phase in which the destruction of cartilage in the osteoarthritis can be recovered to a normal state. That is, the term refers to osteoarthritis in a phase in which the destruction of cartilage is stopped by treatment, meditation, or removal of the causes of the destruction of cartilage, and can be recovered to a normal state of cartilage.

According to a specific embodiment of the present invention, the osteoarthritis in its early stage is in an osteoarthritis progression phase of Grade I to Grade III on classification of Osteoarthritis Research Society International (OARSI). The classification of OARSI is obtained by histologically quantifying the severity of osteoarthritis, the contents of which are disclosed in the document “Osteoarthritis Cartilage 14:13-29, 2006, Pritzker, K. P. et al., Osteoarthritis cartilage histopathology: grading and staging” and are incorporated herein by reference.

In order to improve the usefulness of the peptide of the present invention as a diagnosing reagent, a material generating a detectable signal may be directly or indirectly linked to the peptide of the present invention. The material generating a detectable signal and linked to the peptide of the present invention includes radioisotopes (e.g., C14, I125, P32, and S35), chemicals (e.g., biotin), fluorescent materials [e.g., fluorescein, fluoreeinisothiocyanate (FITC), rhodamine 6G, rhodamine B, 6-carboxy-tetramethyl-rhodamine (TAMRA), Cy-3, Cy-5, Texas Red, Alexa Fluor, 4,6-diamidino-2-phenylindole (DAPI), and coumarin], luminescent materials, chemiluminescent materials, fluorescence resonance energy transfer (FRET) generating materials, and Fe, Gd, Mn, Zn, and lanthanide elements allowing molecular imaging, such as CT, MRI, gamma-camera, SPECT, or PET, but is not limited thereto. Various linkages and linking methods are described in Ed Harlow and David Lane, Using Antibodies: A Laboratory Manual, Cold Spring Harbor Laboratory Press, 1999, the contents of which are incorporated herein by reference.

The material generating a detectable signal may be directly linked to the peptide, and also may be indirectly linked to the peptide. For example, the material generating a detectable signal may be indirectly linked to the peptide, by linking biotin to the peptide and then linking streptavidin (or avidin) combined with the above-described label to the biotin.

In the case where osteoarthritis is detected in vitro or ex vivo by using the peptide of the present invention, the biosample isolated from the living body is used to detect osteoarthritis. In this case, the diagnosing agent of the present invention may be used for conventional immunoassay protocols. The immunological analysis may be carried out according to various quantitative or qualitative immunoassay protocols developed in the prior art. The immunoassay format includes radioactive immunoassay, radioactive immunoprecipitation, immunoprecipitation, enzyme linked immunosorbent assay (ELISA), captured-ELISA, inhibition or competition analysis, sandwich assay, flow cytometry, immunofluorescence, and immunoaffinity purification, but is not limited thereto. The method of immunoassay or immune staining is disclosed in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, NJ, 1984; and Ed Harlow and David Lane, Using Antibodies: A Laboratory Manual, Cold Spring Harbor Laboratory Press, 19, the contents of which are incorporated herein by reference.

In the case where the osteoarthritis is detected in vitro or ex vivo by using the peptide of the present invention, the peptide is directly injected into the living body, and then osteoarthritis is detected. In this case, the detection of osteoarthritis may be carried out using radiography or molecular imaging. The molecular imaging technique usable herein includes optical imaging, computed tomography (CT), and magnetic resonance imaging (MRI), but is not limited thereto.

In accordance with an aspect of the present invention, there is provided a kit for use in detecting osteoarthritis of a subject comprising a peptide probe containing (i) a peptide comprising the amino acid sequence of SEQ ID NO: 1 and (ii) a label generating a detectable signal and linked to the peptide in the subject or the chondrocytes.

A kit of the present invention uses a peptide probe containing a peptide comprising the amino acid sequence of SEQ ID NO: 1 and a label. Therefore, the overlapping descriptions there between are omitted to avoid excessive complication of the specification due to repetitive descriptions thereof.

### Advantageous Effects

Features and advantages of the present invention are summarized as follows: The present invention relates to a reagent for diagnosing osteoarthritis or predicting the prognosis of osteoarthritis, the reagent containing a peptide having the amino acid sequence (CQRPPR) of SEQ ID NO: 1 as an active ingredient. The peptide of the present invention can be used to accurately diagnose osteoarthritis in its early stage based on the molecular imaging technique. The peptide of the present invention has a small molecular weight, and thus has advantages of fast clearance from the blood, effective permeation into the tissue, low immunogenicity, and low-production cost. Further, the reagent of the present invention can diagnose osteoarthritis in its early stage in which the destruction of cartilage is in a reversible phase and thus can be recovered to a normal state, thereby significantly contributing to effective treatment of osteoarthritis.

### BRIEF DESCRIPTION OF THE DRAWINGS

The application file contains drawings executed in color (FIGS. 1-6). Copies of this patent or patent application with color drawings will be provided by the Office upon request and payment of the necessary fee.

FIG. 1 shows morphogenic and histology analyses during OA progression. Panel A indicates morphogenic pictures of arthritic mouse joints at different stages of arthritis development following DMM operation. Panel B indicates Safranin O-stained sections of normal and arthritic mouse joints at different stages of arthritis development following DMM operation.

FIG. 2 shows in vivo and ex vivo optical images of OA progression. FIG. 2A indicates in vivo optical images of DMM mice after intravenous injection of ApoPep-1 probe at different stages of arthritis development (1, 2, 4, and 8 weeks after DMM operation) (n=6-10 each group). FIG. 2B indicates ex vivo optical images of DMM mice after intravenous injection of ApoPep-1 probe at different stages of arthritis development (2, 4, and 8 weeks after DMM operation) (n=3 each group). FIG. 2C-2D indicates that data are mean±SD.*p<0.01 01 compared to Sham-ApoPep-1 groups, #p<0.01 compared to Sham groups.

FIG. 3 shows in vivo optical images of ApoPep-1 probe in DMM micemodel. FIG. 3A-3B indicates in vivo optical images of normal and DMM mice after intravenous injection of ApoPep-1 probe. DMM model was made as shown in Materials and Methods. Positive ApoPep-1 signals were detected strongly at 2 weeks after operation in DMM model. Data are mean±SD. **p<0.01 compared to Sham-ApoPep-1. #p<0.01 compared to OA-Control Peptide.

FIG. 4 shows ApoPep-1 binding to apoptotic chondrocytes in TUNEL assay. The figure shows ApoPep-1 staining of the OA cartilage in DMM and sham-operated mice at 2 weeks after surgery. Immunofluorescent staining was performed in cryo-sections with TUNEL (red) and ApoPep-1 (yellow) as described in Materials and Methods.

FIG. 5 shows expression pattern of type II collagen, MMP 13, and ApoPep-1 positive signals in the OA and control cartilage. FIG. 5 indicates staining of various markers in OA cartilage and sham-operated control mice at 2 weeks after surgery. Immunofluorescence staining was performed in cryo-sections with Col 2 (red), MMP13 (green) and ApoPep-1 (yellow) as described in Materials and Methods. Scale bar, 50 μm.

FIG. 6. shows in vitro binding of ApoPep-1 to apoptotic chondrocyte. ATDC5 cells were incubated with Staurosporine (0.5 μM) for 6 hours to induce apoptosis (FIG. 6A). Cells were stained with annexin V (green), ApoPep-1 (red), and DAPI (blue). Magnification, ×40 (FIG. 6B).

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### EXAMPLES

### Materials and Methods

### Example 1

### Experimental Animals and Surgical Procedures

C57BL/6N mice were purchased from KOATECH (Gyeonggi-do, Republic of Korea), and animal care and experiments were carried out in accordance with the Institutional Animal Care and Use Committees of Kyungpook National University (KNU 2011-68). Animals were maintained on a 12 hours light: 12 hours darkness cycle at 22-25° C. in specific pathogen-free conditions and fed with standard rodent chow and water ad libitum.

Male mice at 12 weeks of age were divided into three groups: Sham-ApoPep-1, OA-Control Peptide and OA-ApoPep-1 group (n=4-8/group). For OA model, we performed DMM surgery as described in previous studies (8, 23).

### Example 2

### Safranin-O Staining and Histological Analysis

The decalcified tissues dehydrated with an increasing concentration of ethanol and embedded in paraffin, and then sectioned with a thickness of 3 μm. For Safranin O staining, sections were deparaffined and rehydrated then dipped into Weigert's iron hematoxylin (sigma Aldrich) for 10 min, fast green solution (sigma Aldrich) for 5 minutes, and 0.1% Safranin O solution (sigma Aldrich) for 5 min. OA development in the tibia plateau was quantified by histological grading scores of 0-4 for cartilage destruction (8).

### Example 3

### Optical In Vivo and Ex Vivo Imaging

FlammaTM675-ApoPep-1 was intravenously (i.v.) injected from the tail vain (1 mM 100 μl/20 g) and allowed to circulate for the indicated time periods. In vivo optical imaging was conducted by scanning the mice under anesthesia by inhalation of isofluran (jw pharmaceutical, South Korea) in 80% N2O/20% O2 using Optix Explore (ART, Montreal, Canada). The excitation/emission wavelengths for FlammaTM675 were 676 nm/704 nm. Ex vivo optical imaging was performed with extracted hind limbs by the same method. FlammaTM675-NSSSVDK was used as the control peptide (18).

### Example 4

### Immunofluorescence Staining

Hind limbs of mice were fixed with 4% paraformaldehyde in 0.1 M phosphate-buffered saline (PBS, pH 7.4) for 12 hours, and decalcified with 10% ethylene-diamine tetraacetic acid (EDTA, pH 7.4) for 3 weeks. Decalcified tissues were dipped in 20% sucrose/PBS solution overnight and embedded in OCT compound (Tissue-Tek) for making the frozen samples. Frozen samples were cut at 10 μm thickness from femur to tibia range for joint visible. Cut slices from each animal were selected for the staining with same primary antibodies to comparison at the same area of joint articular cartilage. The section was treated with a blocking solution (0.1% tween-20, 1% bovine serum albumin (BSA), 5% normal donkey serum in PBS) after washing with PBS, and then incubated at 4° C. 1 h with the primary antibodies such as rabbit polyclonal collagen 2 antibody (1:200; abcam, MA, USA), mouse monoclonal MMP-13 antibody (1:200; abcam, MA, USA). After PBS rinses, the sections were incubated with Alexa-594 or Alexa-488-conjugated secondary antibody for 30 min. Thereafter, the sections were incubated with 4′,6′-diamidino-2-phenylindole (DAPI, Sigma-Aldrich, St Louis, Mo., USA) for nuclear staining then mounted with a ProLong® Antifade Kit (Invitrogen, USA). Images of the stained sections were taken pictures using a Zeiss LSM-510 Meta confocal microscope (Zeiss, Oberkochen, Germany), all pictures were taken by microscope operator.

### Example 5

### TUNEL Assay

TUNEL assay was conducted using an ApopTag® Red In Situ Apoptosis Detection Kit (Millipore, USA & Canada) according to the manufacturer's instructions. Frozen sections were air-dried for 30 min at room temperature and washed 3 times with PBS/3 min. The sections were pre-fixed by 1% PFA for 10 min at room temperature, subsequently to post-fixation by precooled ethanol/acetic acid (2:1) for 5 min at −20° C. The fixed tissue sections were applied in equilibration buffer for 10 seconds at room temperature, and were reactive in TdT enzymes for 30 min in 37° C. then incubated with DAPI for nuclei staining and mounted with a ProLong® Antifade Kit. Images of the stained sections were taken pictures using a Zeiss LSM-510 Meta confocal microscope, all picture were taken by microscope operator.

### Example 6

### Cell Culture and Immunocytochemistry

ATDC5 chondrocyte cell was maintained in a mixture of DMEM and Ham's F-12 (DMEM/F12) medium (Lonza, USA) containing 5% fetal bovine serum (FBS) (Gibco-BRL, USA), 10 ug/ml of human transferrin (Sigma) and 3×10-8 M of sodium selenite (Sigma) and penicillin/streptomycin. Apoptosis was induced by incubating cells with 0.5 uM Staurosporine (cell signaling, USA) for the indicated time periods. Apoptotic stages were determined by staining the cells with annexin V and ApoPep-1 conjugated with flamma 675. For immunocytochemistry staining, cells were incubated with 1% bovine serum albumin at 37° C. for 30 min for blocking and then binding with 10 μM flamma 675-conjugated peptide at 4° C. for 1 h. Cells were then costained with Alexa-594-annexin V (Invitrogen, Carlsbad, Calif., USA) for 15 min at room temperature in binding buffer (10 mM HEPES, pH 7.4, 140 mM NaCl, and 2.5 mM CaCl2). After fixation, cells were incubated with DAPI for nuclear staining and mounted with a ProLong® Antifade Kit.

### Example 7

### Statistics

A statistical analysis was performed using SigmaPlot software. Data were expressed as group mean±SD. Data from the analyzed experiments were using sigma Plot followed by t′ test. A value of p<0.05 was considered statistically significant.

### Results

1. Efficacy of OA Surgery

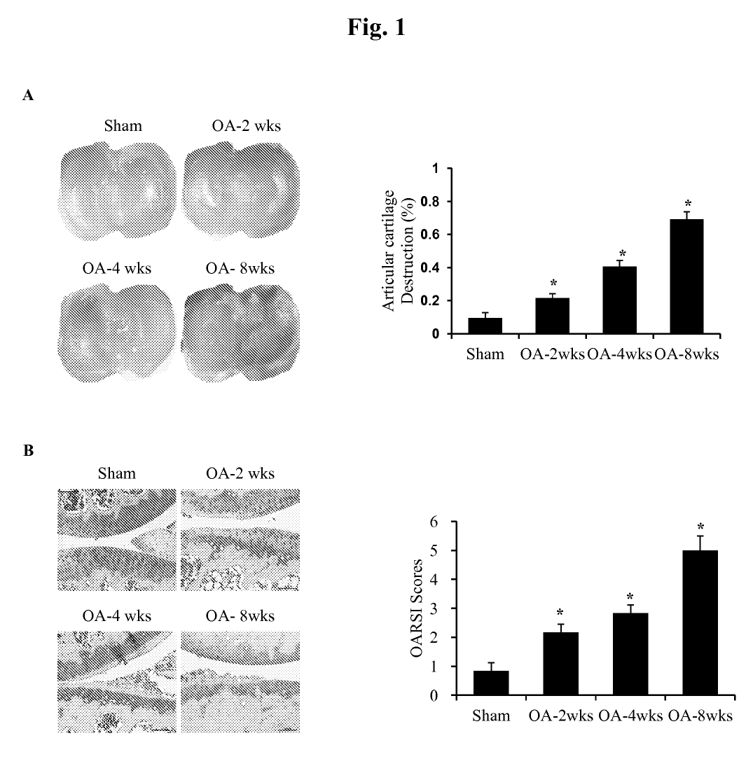

We compared the effect of ApoPep-1 conjugated with a fluorescent dye to assess OA cartilage in surgically-induced OA mice. Morphological and histological analyses revealed that sham mice had regular cartilage surfaces with clear boundaries between the cartilage and calcified bone surfaces throughout the experiment (FIG. 1). However, joints of mice that received the destabilization of the medial meniscus (DMM) surgery matched the description of grade I OA at 2 weeks after surgery, grade III OA at 4 weeks post-surgery, and grade VI OA at 8 weeks post-surgery, according to the Osteoarthritis Research Society International (OARSI) diagnosis criteria. According to these criteria, grade I and II denote that the superficial zone remains intact, although there may be some microscopic fibrillation and fissuring, and the middle and deep zones are unaffected. Grade III changes appear when vertical fissures extend into the middle zone, but there is still no significant cartilage loss. Grades I to III depict early OA, when the disease is thought to be potentially reversible. Thus, in order to prevent the progression of OA, OA should be diagnosed no later than grade III OA, thus by 4 weeks post-DMM surgery in our experimental mice. Grade IV OA develops when increased fissuring results in cartilage erosion. Grade V and VI OA describe almost complete erosion of the articular cartilage with changes affecting the underlying bone, such as sclerosis and eburnation.

2. ApoPep-1 Detects OA Cartilage In Vivo

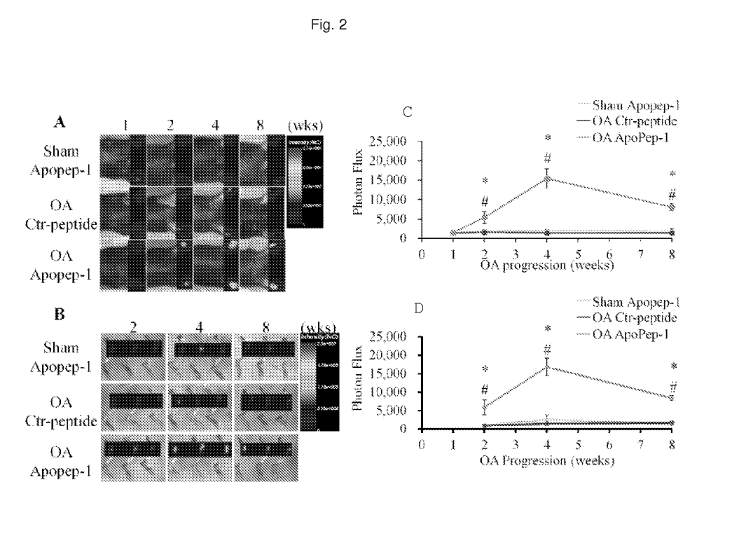

Optical images of the fluorescence-labeled ApoPep-1 probe were clearly detectable from arthritic joints from 30 minutes to 4 hours after injection (FIG. 2). Compared with the sham-ApoPep-1 and OA-control-Peptide group mice, there was no significant difference at one week, while a significant increase of ApoPep-1 fluorescent signal was observed from the arthritic joints at two weeks, and it was maximal at 4 weeks in vivo (FIGS. 2A and 2C) and ex vivo (FIGS. 2B and 2D). However, the ApoPep-1 fluorescent signal from the arthritic joints was reduced at 8 weeks compare with at 4 weeks, despite more cartilage destruction at 8 weeks. These results clearly indicate that ApoPep-1 probe can detect apoptotic change at least at 2 weeks after in DMM OA model.

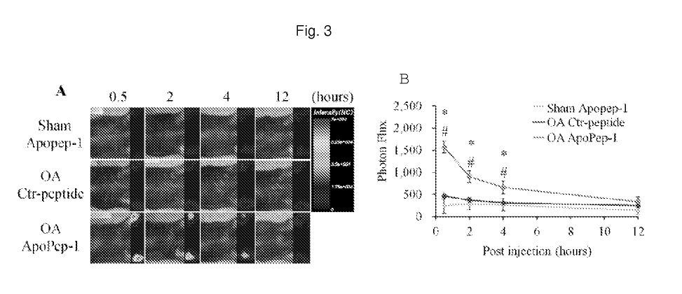

To determine whether fluorescence-labeled ApoPep-1-probe could be used for the early diagnosis of OA and to monitor the progression of OA more precisely, we observed fluorescent images at different stages of OA development in DMM mice. First, optical images were obtained successively from 30 minutes to 24 hours after administering the probe with DMM models intravenously (FIG. 3A). Fluorescent signal was clearly detectable from arthritic joint sat 30 minutes after the probe injection and decreased according to time course (FIG. 3B). These results indicated that the optimal time point for diagnosis was 30 minutes after injection of ApoPep-1 probe.

Taken together, these results indicate that ApoPep-1 could be a useful imaging probe for detecting early stage of OA disease.

3. Histologic Analysis of ApoPep-1 Binding in the OA

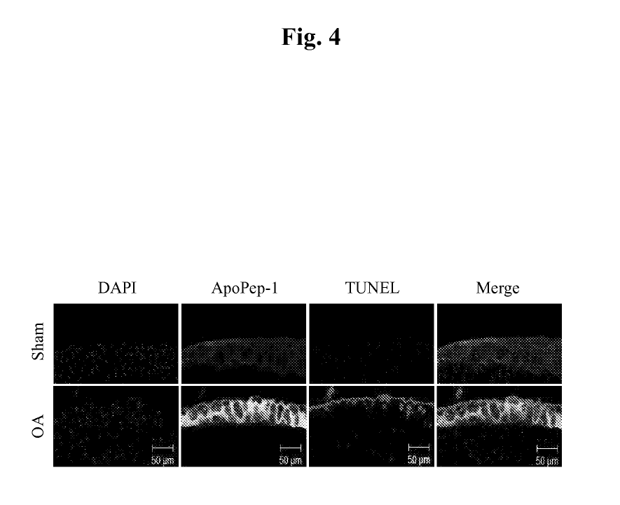

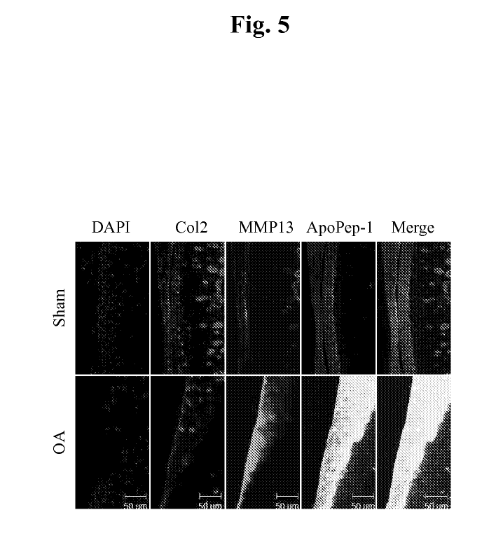

The arthritic joints were analyzed by in situ immunofluorescence staining to confirm the correlation of positive ApoPep-1 signals and molecular imaging signals at 2 weeks post operation. First, we tested whether ApoPep-1 binds to apoptotic chondrocytes. Terminal deoxynucleotidyltransferase-mediated dUTP nick-end labeling (TUNEL) staining, which detects fragmented DNA and thus senses apoptotic cells, was also strongly correlated with ApoPep-1 signals in DMM models 2 weeks after surgery (FIG. 4). Apoptotic chondrocytes lose their ability to synthesize type II collagen [15] and increase the secretion of matrix degrading enzymes such as matrix metalloproteinase 13 (MMP13). The death of chondrocytes in patients with knee OA are characterized by an uncoupling of type II collagen synthesis and degradation of collagen fibrils in the extracellular matrix and increased MMP13. Our results show that ApoPep-1 was detected where type II collagen decreased and MMP13 increased in DMM-operated articular cartilage (FIG. 5). These in situ results suggest that ApoPep-1 binds to apoptotic cells of degenerative cartilage area in mice as early as 2 weeks after DMM surgery (grade I OA).

4. In Vitro Binding of ApoPep-1 to Apoptotic Cells

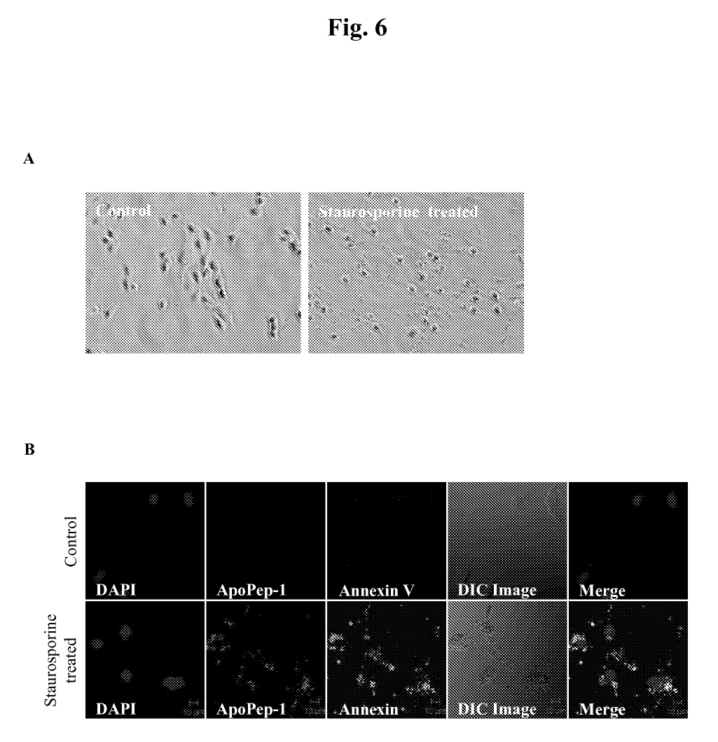

ApoPep-1 binds to apoptotic chondrocytes in vitro. To better define the binding ability of ApoPep-1 to apoptotic cells, we used chondrocytes treated with staurosporine, which induces shrinkage and apoptosis of cells. In vitro fluorescence staining revealed that ApoPep-1 and the apoptosis marker annexin V both bind to apoptotic cells, but not to live cells (FIG. 6). These results support that ApoPep-1 binds to apoptotic chondrocytes.

### DISCUSSION

In this study, we found that ApoPep-1 probe can be used as a diagnostic tool to detect early stage of OA. Fluorescent ApopPep-1 is a noninvasive molecular probe specifically targeted to apoptotic cells in DMM OA models and applicable to a valuable therapeutic prognosis monitoring as well as early diagnosis of OA.

The optical images of ApoPep-1 probe, both in vivo and ex vivo, dominantly increased in the OA groups. The OARSI grading histologically scores the severity of OA (24). Grade I and II changes describe cartilage edema and the condensation of collagen fibers with early glycosaminoglycan depletion. The superficial zone remains intact, although there may be some microscopic fibrillation and fissuring, and the middle and deep zones are unaffected. Grade III changes are seen when vertical fissures extend into the middle zone but there is still no significant cartilage loss. Grades I to III depict early OA, when the disease is thought to be potentially reversible. Grade IV OA develops when increased fissuring results in cartilage erosion. Grade V and VI OA describe almost complete erosion of the articular cartilage, with changes affecting the underlying bone, such as sclerosis and eburnation (25). At the 2 weeks after DMM operation, our DMM model showed slight glycosaminoglycan depletion and little pathogenic changes, which are early stage of OA progression in Grades I. ApoPep1 probe detected apoptotic chondrocytes in Grade II stage of OA in DMM model, indicating within the reversible stage. ApoPep-1 binding was maximal after 30 minutes after injection and then it was decreasing and undetectable after 8 hours. It has been known that ApoPep-1 targets histone H 1.2 which is exposed in apoptotic cells surface of apoptotic tumor cells as well as neuronal cells of brain (22, 26). Human OA along with aging shows chondrocyte apoptosis in articular cartilage (27, 28). Indeed, TUNEL positive cell areas showed the high binding of ApoPep-1 probe, suggesting. ApoPep-1 can detect apoptotic chondrocytes.

In the early stage of OA, the superficial zone of articular cartilage was found to be cells biologically defective without prominent changes like a decrease of Safranin-O positive proteoglycan production (8). As expected, slight loss of Safranin-O staining and reduction of the type II collagen production were observed in articular chondrocytes at early stage of OA in DMM model. Apoptotic chondrocytes lost their ability to synthesize type II collagen (29, 30), while they increased matrix degradation enzymes such as MMP13 [6-8]. We showed that the in vivo results were recapitulated with in vitro experiments using chondrocyte cell line ATDC5 showing the binding of ApoPep-1 probe to apoptotic chondrocytes.

The development of preventative strategies for OA requires the ability to identify OA at a stage when cartilage degradation is reversible and the point-of return exists. Grades I to III depict early OA, when the disease is thought to be potentially reversible. ApoPep-1 probe can detect the OA development from the Grades I which is at 2 weeks after DMM surgery. These data provide a clue when cartilage degradation is reversible stage ApoPep-1 probe can detect the early OA.

Collectively, we highlight ApoPep-1 probe facilitated early diagnosis of OA within reversible period, suggesting a new rapid in vivo OA diagnosis method as well as valuable approach for monitoring of OA therapeutic prognosis and prevention.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method for detecting osteoarthritis of a subject comprising:
(a) administering to the subject or chondrocytes obtained from the subject a peptide probe containing (i) a peptide comprising the amino acid sequence of SEQ ID NO: 1 and (ii) a label generating a detectable signal and linked to the peptide;
(b) determining the level of the peptide in the subject or the chondrocytes by measuring signal generated from the peptide probe; and
(c) comparing the level of the peptide in the subject or chondrocytes, with the level of the peptide in a normal subject or normal chondrocytes, wherein an increased level of the peptide in the subject indicates an increased severity of osteoarthritis of the subject.

2. The method according to claim 1, wherein the subject is a human, a mouse, a rat, a hamster, a rabbit, a guinea pig, a dog or a primate.

3. The method according to claim 1, wherein the peptide binds to the chondrocytes.

4. The method according to claim 1, wherein the chondrocytes are apoptotic chondrocytes.

5. The method according to claim 1, wherein osteoarthritis is in its early stage.

6. The method according to claim 5, wherein osteoarthritis in its early stage is characterized by destruction of a cartilage within a reversible phase.

7. The method according to claim 5, wherein osteoarthritis in its early stage is in an osteoarthritis progression phase of Grade I to III based on classification of OARSI (Osteoarthritis Research Society International).

8. The method according to claim 1, wherein the label is a radioactive isotope, a fluorescence material, a chemiluminescence material, a chromogenic enzyme, or a FRET (fluorescence resonance energy transfer)-generating material.

9. The method according to claim 1, wherein the determination of the level of the peptide in the step (b) is performed at from 20 minutes to 24 hours after administering the peptide probe.

10. The method according to claim 1, wherein the determination of the level of the peptide in the step (b) is performed at 30 minutes after administering the peptide probe.

11. A kit for use in detecting osteoarthritis of a subject according to claim 1, comprising a peptide probe containing (i) a peptide comprising the amino acid sequence of SEQ ID NO: 1 and (ii) a label generating a detectable signal and linked to the peptide in the subject or the chondrocytes.


SEQUENCE LISTING (1 sequence)

SEQ ID NO: 1 - protein, 18 aa, Artificial Sequence
  Synthetic construct
ALAALAALAALAALAALA